(19) United States
(12) Patent Application Publication
(10) Pub. No.: US 2018/0228716 A1
(43) Pub. Date: Aug. 16, 2018

(54) Essence of alayah - hair seen

(71) Applicant: James Edward Mclean, JR., Charlotte, NC (US)

(72) Inventor: James Edward Mclean, JR., Charlotte, NC (US)

(73) Assignee: Mcleanics Technology Corporation

(21) Appl. No.: 15/530,642

(22) Filed: Feb. 14, 2017

(51) Int. Cl. A61K 8/891; A61Q 5/00; A61Q 5/12; A61K 8/67

(52) U.S. Cl. CPC A61K 8/891; A61Q 5/00; A61K 2800/805; A61K 8/678; A61Q 5/12

(57) ABSTRACT

The Essence Of Alayah—Hair Seen (EOA), is a hair sheen or hair polish that is specially designed and formulated to add a brilliant sheen, gloss, or shine to any type hair style, and may be used to as a final touch to top off a hair style. The EOA consist of a chemical mixture of Cyclomethicone and Dimethicone, and/or any other silicone or oil based chemical mixture; and said chemical mixture has a final viscosity approximately equal to that of water, or a viscosity that enables the chemical mixture to be sprayed as a mist onto the hair and not weigh down the hair as does most oil sprays. The EOA may contain Vitamin “E” as part of its chemical mixture, or other vitamins to help promote healthier hair and hair growth. Further, the EOA will incorporate either of the following fruit fragrances, floral fragrances, or candy fragrances: Sweet Cherry, Cherry, Sweet Apple, Apple, Sweet Rose, Rose, Sweet Bubble Gum, Bubble Gum, Sweet Peach, Peach, Sweet Strawberry, Strawberry, Sweet Balsam, Balsam, Sweet Pineapple, Pineapple, Sweet Banana, Banana, Sweet Honeysuckle, Honeysuckle. Sweet Pear, Pear, Sweet Coconut, Coconut, Sweet Mango or Mango. And, the EOA may be used as a conditioner aid. As of today, most hair care products that add sheen to the hair incorporates some type of oil that when applied to the hair will weigh down the hair or give the hair a dull shine, the EOA solves this problem.

### CROSS REFERENCE TO RELATED APPLICATION

This Application is related to the Essence Of Alayah—Hair Seen Provisional Patent Application, Application No. 62/387,935; filed on Jan. 12, 2016.

### STATEMENT REGARDING FEDERAL SPONSORED RESEARCH

None Applicable.

### REFERENCE TO SEQUENCE LISTING, TABLE, OR A COMPUTER PROGRAM LISTING COMPACT DISC APPENDIX

None Applicable.

### BACKGROUND OF INVENTION

The Essence Of Alayah—Hair Seen (EOA), is in the field of hair care products. As of today, most hair care products that add sheen to the hair incorporates some type of oil that when applied to the hair will weigh down the hair or give the hair a dull shine, the EOA solves this problem. The EOA is specially designed and formulate to add a brilliant sheen, gloss, or shine to any type hair styles, and may be used as a final touch to top off a hair style without weighing down the hair. And, the EOA may be used as a hair conditioner aid.

### BRIEF SUMMARY OF INVENTION

The Essence Of Alayah—Hair Seen (EOA), is a hair sheen or hair polish that is specially designed and formulated to add a brilliant sheen, gloss, or shine to any type hair style, and may be used as a final touch to top off a hair style. The EOA consist of a chemical mixture of Cyclomethicone and Dimethicone, and/or any other silicone or oil based chemical mixture; and said chemical mixture has a final viscosity approximately equal to that of water, or a viscosity that enables the chemical mixture to be sprayed as a mist onto the hair and not weigh down the hair as does most oil sprays. The EOA may contain Vitamin “E” as part of its chemical mixture, or other vitamins to help promote healthier hair and hair growth. Further, the EOA will incorporate either of the following fruit fragrances, floral fragrances, or candy fragrances: Sweet Cherry, Cherry, Sweet Apple, Apple, Sweet Rose, Rose, Sweet Bubble Gum, Bubble Gum, Sweet Peach, Peach, Sweet Strawberry, Strawberry, Sweet Balsam, Balsam, Sweet Pineapple, Pineapple, Sweet Banana, Banana, Sweet Honeysuckle, Honeysuckle, Sweet Pear, Pear, Sweet Coconut, Coconut, Sweet Mango or Mango.

### BRIEF DESCRIPTION OF THE SEVERAL VIEWS OF THE DRAWINGS

FIG. 1, is the spray bottle type packaging of the Invention.

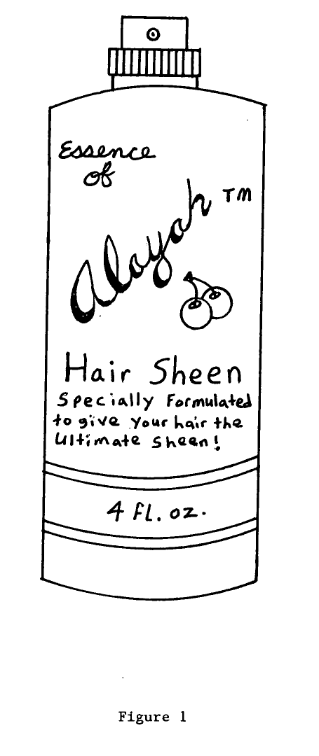

FIG. 2, is the formula arrangement of the Invention.

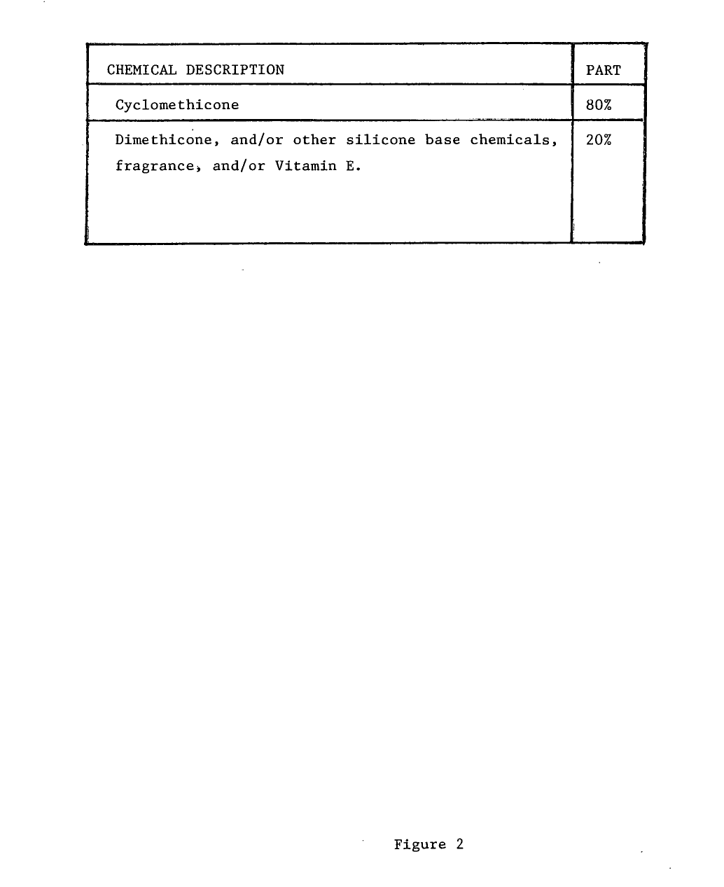

### DETAIL DESCRIPTION OF THE INVENTION

The Essence Of Alayah—Hair Seen (EOA), is a hair sheen or hair polish that is specially designed and formulated to add a brilliant sheen, gloss, or shine to any type hair style, and may be used as a final touch to top off a hair style. The EOA consist of a chemical mixture of Cyclomethicone and Dimethicone, and/or any other silicone or oil based chemical mixture; and said chemical mixture has a final viscosity approximately equal to that of water, or a viscosity that enables the chemical mixture to be sprayed as a mist onto the hair and not weigh down the hair as does most oil sprays. The EOA may contain Vitamin “E” as part of its chemical mixture, or other vitamins to help promote healthier hair and hair growth. Further, the EOA will incorporate either of the following fruit fragrances, floral fragrances, or candy fragrances: Sweet Cherry, Cherry, Sweet Apple, Apple, Sweet Rose, Rose, Sweet Bubble Gum, Bubble Gum, Sweet Peach, Peach, Sweet Strawberry, Strawberry, Sweet Balsam, Balsam, Sweet Pineapple, Pineapple, Sweet Banana, Banana, Sweet Honeysuckle, Honeysuckle, Sweet Pear, Pear, Sweet Coconut, Coconut, Sweet Mango or Mango.

In addition, the EOA will help to protect the hair from damage, which could result from the lost of natural oils during shampooing.

Finally, the EOA may serve as a hair conditioner aid by providing softness and ease manageability; thus, helping to prevent dry and brittle hair. The EOA may be applied directly to the hair as well as sprayed onto the hair; therefore, enabling the EOA to act as a conditioner aid.

We claim:

1) A Chemical mixture, sprayed on or applied to the hair to add a sheen, gloss, or shine to the hair; wherein said chemical mixture consist of Cyclomethicone and Dimethicone, or some other type of silicone or oil base chemical mixture, with the viscosity equal to or approximately equal to that of water, or a viscosity so as to enable the chemical mixture to be sprayed as a mist, and said chemical mixture incorporates a floral, fruit, or candy scent, and incorporates vitamin E or other type of vitamins.

2) The chemical mixture, as outlined in claim number 1, wherein said floral scent is Sweet Rose or Rose.

3) The chemical mixture, as outlined in claim number 1, wherein said floral scent is Sweet Balsam or Balsam.

4) The chemical mixture, as outlined in claim number 1, wherein said fruit scent is Sweet Apple or Apple.

5) The chemical mixture, as outlined in claim number 1, wherein said fruit scent is Sweet Cherry or Cherry.

6) The chemical mixture, as outlined in claim number 1, wherein said candy scent is Sweet Bubble Gum or Bubble Gum.

7) The chemical mixture, as outlined in claim number 1, wherein said fruit scent is Sweet Strawberry or Strawberry.

8) The chemical mixture, as outlined in claim number 1, wherein said fruit scent is Sweet Peach or Peach.

9) The chemical mixture, as outlined in claim number 1, wherein said fruit scent is Sweet Coconut or Coconut.

10) The chemical mixture, as outlined in claim number 1, wherein said fruit scent is Sweet Pear or Pear.

11) The chemical mixture, as outlined in claim number 1, wherein said fruit scent is Sweet Banana or Banana.

12) The chemical mixture, as outlined in claim number 1, wherein said fruit scent is Sweet Pineapple or Pineapple.

13) The chemical mixture, as outlined in claim number 1, wherein said floral scent is Sweet Honeysuckle or Honeysuckle.

14) The chemical mixture, as outlined in claim number 1, wherein said fruit scent is Sweet Mango or Mango.
